(19) United States
(12) Patent Application Publication
(10) Pub. No.: US 2026/0071260 A1
(43) Pub. Date: Mar. 12, 2026

(54) CELL-FREE TOTAL NUCLEIC ACID CONTROLS

(71) Applicant: Microgenics Corporation, Fremont, CA (US)

(72) Inventors: Kunal Banjara, Hayward, CA (US); Liang-Chun Liu, Cupertino, CA (US); Qiang Gan, Fremont, CA (US)

(73) Assignee: Microgenics Corporation, Fremont, CA (US)

(21) Appl. No.: 19/107,217

(22) PCT Filed: Aug. 28, 2023
(86) PCT No.: PCT/US2023/072998

§ 371 Date: Feb. 27, 2025

Related U.S. Application Data
(60) Provisional application No. 63/402,027, filed on Aug. 29, 2022.

(51) Int. Cl. C12Q 1/6827; C12N 15/11; C12Q 1/6886

(52) U.S. Cl. CPC C12Q 1/6827; C12N 15/11; C12Q 1/6886; C12Q 2600/166

(57) ABSTRACT

Cell-free total nucleic acid (cfTNA) compositions including genomic DNA, one or more DNA molecules including one or more DNA variants, and one or more RNA molecules including one or more RNA variants are provided. Also provided are kits including the cfTNA compositions. In addition, methods of making and using the cfTNA compositions are provided.

### CROSS REFERENCE TO RELATED APPLICATIONS

This application claims the benefit of U.S. Provisional Application No. 63/402,027, filed Aug. 29, 2022, which is incorporated herein by reference in its entirety.

### FIELD

This disclosure relates to cell-free total nucleic acid compositions and methods of use, particularly as controls in kits and methods of determining biomarkers in a sample.

### BACKGROUND

The use of Next Generation Sequencing (NGS) assays for early cancer diagnosis and disease monitoring has become a critically important aspect of personalized medicine. Most NGS assays have focused on detection of either DNA or RNA variants from patient samples (including solid tumor fresh frozen or FFPE samples). Recent studies also include the use of liquid biopsy samples, including plasma, serum, saliva, CSF, or other body fluids that contain genetic information from both normal and cancerous cells. Useful genetic information can be gleaned from both DNA and RNA; however, existing assays are typically capable of detecting only one type of nucleic acid, not both. Similarly, quality control materials that mimic liquid biopsy are also limited, generally containing either DNA or RNA only.

### SUMMARY

There is a need for quality control materials including both DNA and RNA. Disclosed herein are materials that mimic cell-free total nucleic acid (cfTNA) with both DNA and RNA representing genes from either normal or disease state cells, and methods of their preparation and use.

Provided herein is a cell-free total nucleic acid (cfTNA) composition that includes: a) genomic DNA fragments of about 75 to about 600 bp in length, b) one or more DNA molecules including one or more variants selected from the group consisting of one or more single nucleotide polymorphisms (SNP), single nucleotide variants, substitution of one or more nucleic acids (e.g., point mutations), insertions (INS), deletions (DEL), premature stop codons, trinucleotide repeats, translocations, inversions, somatic rearrangements, allelomorphs, splice variants, regulatory variants, gene fusions, copy number variation (CNV), and any combination of two or more thereof, and c) one or more RNA molecules including one or more variants selected from fusions, isoforms, splice variants, and any combination thereof.

In some examples, the genomic DNA is human genomic DNA. The genomic DNA fragments may be about 200 bp in length in some examples. In particular examples, the composition includes about 40 ng/μL to about 60 ng/μL genomic DNA.

In some examples, at least one of the DNA molecules in the cfTNA composition includes two or more variants. In particular examples, at least one of the one or more DNA molecules is present in the composition at a variant allelic frequency of about 0.5% to about 1.5%. In further examples, at least one of the one or more DNA molecules is present in the composition at a copy number of about 4 to about 7. In some examples, the one or more DNA molecules are selected from the group consisting of plasmid DNA, bacterial artificial chromosome DNA, double-stranded DNA fragments (for example, gBlock DNA fragments), or any combination of two or more thereof.

In some examples, at least one of the one or more RNA molecules in the cfTNA composition includes in vitro transcribed RNA. At least one of the RNA molecules may be present in the composition at a copy number of about 25 to about 125 copies.

In some examples, at least one of the DNA variants included in the cfTNA compositions, at least one of the RNA variants included in the cfTNA composition, or both include a variant associated with a disease or disorder. In some examples, the disease or disorder is cancer. In other examples, the disease or disorder is a genetic disorder.

The cfTNA composition may be formulated in an aqueous solution in some examples. In one example, the aqueous solution is a nucleic acid dilution solution.

Also provided are kits including the cfTNA compositions described herein. The kits may also include one or more enzymes, buffers, oligonucleotide primers, oligonucleotide adaptors, oligonucleotide probes, or any combination of two or more thereof.

Provided herein are methods of making the disclosed cfTNA compositions. The methods include combining: a) a population of genomic DNA fragments of about 75 to about 600 bp in length, b) one or more DNA molecules comprising one or more variants selected from the group consisting of one or more single nucleotide polymorphism (SNP), single nucleotide variants, substitution of one or more nucleic acids (e.g., point mutations), insertions (INS), deletions (DEL), premature stop codons, trinucleotide repeats, translocations, inversions, somatic rearrangements, allelomorphs, splice variants, regulatory variants, gene fusions, copy number variations (CNV), and any combination of two or more thereof, and c) one or more RNA molecules comprising one or more variants selected from fusions, isoforms, and any combination thereof.

In some examples, the population of genomic DNA fragments is prepared by fragmenting a preparation of total genomic DNA and purifying fragments of about 75 to about 600 bp in length. In some examples, the genomic DNA fragments are about 200 bp in length. The genomic DNA may be human genomic DNA.

In some examples, the one or more DNA molecules comprise one or more of plasmid DNA, bacterial artificial chromosome (BAC) DNA, double-stranded DNA fragments, or any combination of two or more thereof. The method may further include fragmenting one or more plasmid DNA or BAC DNA including one or more variants and purifying fragments of about 100 to about 500 bp in length.

In some examples, the method also includes preparing the one or more RNA molecules including the one or more variants by in vitro transcription from one or more DNA molecules encoding the RNA.

In additional examples, the methods further include measuring the concentration of the genomic DNA fragments, the one or more DNA molecules, and/or the one or more RNA molecules, determining the copy number of the genomic DNA fragments, the one or more DNA molecules, and/or the one or more RNA molecules, or both. In further examples, the methods include mixing each of the genomic DNA fragments, the one or more DNA molecules, and/or the one or more RNA molecules at a selected concentration or copy number. In some examples, the methods include combining the genomic DNA fragments, the one or more DNA molecules, and the one or more RNA molecules in an aqueous solution.

The foregoing and other features of the disclosure will become more apparent from the following detailed description, which proceeds with reference to the accompanying figures.

### BRIEF DESCRIPTION OF THE DRAWINGS

FIG. 1 is a schematic diagram showing an exemplary method for preparing a cfTNA control of the disclosure. The includes fragmentation and purification of human genomic DNA (here exemplified by GM24385), plasmid DNA, and BAC clone DNA, and linearization and in vitro transcription (IVT) of plasmid DNA to prepare RNA. Each of these preparations, and additional double-stranded DNA fragments (such as gBlocks™ fragments are analyzed for copy number (such as using droplet digital PCR (ddPCR), then combined to produce the cfTNA control.

FIGS. 2A and 2B illustrate gDNA fragmentation pattern tested on TapeStation (FIG. 2A) and BioAnalyzer (FIG. 2B).

FIG. 3 is a plot showing variant allelic frequency for the indicated DNA variants. DNA variants are indicated on the x-axis, and y-axis is the variant allelic frequency (VAF) measured in each library replicate. NGS libraries from different lots of cfTNA controls are indicated with different shading.

FIG. 4 is a plot showing molecular counts for the indicated fusion isoforms. RNA fusions are indicated on the x-axis, and y-axis is the molecular counts per 30 ng. NGS libraries from different lots of cfTNA controls are indicated with different shading.

### DETAILED DESCRIPTION

### I. Terms

Section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc., discussed in the present teachings, such that slight and insubstantial deviations are within the scope of the present teachings herein. In this application, the use of the singular includes the plural unless specifically stated otherwise. It is noted that, as used in this specification, singular forms “a,” “an,” and “the,” and any singular use of a word, include plural referents unless expressly and unequivocally limited to one referent. Also, the use of “comprise,” “comprises,” “comprising,” “contain,” “contains,” “containing,” “include,” “includes,” and “including” are not intended to be limiting. It is to be understood that both the general description is exemplary and explanatory only and not restrictive of the disclosure.

Unless otherwise defined, scientific and technical terms used in connection with the disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization used herein are those well-known and commonly used in the art. The practice of the present subject matter may employ, unless otherwise indicated, techniques and descriptions used in organic chemistry, molecular biology (including recombinant techniques), cell biology, and biochemistry, which are within the skill of the art. Such techniques include, but are not limited to, preparation of synthetic polynucleotides, polymerization techniques, chemical and physical analysis of polymer particles, preparation of nucleic acid libraries, nucleic acid sequencing and analysis, and the like. Specific illustrations of suitable techniques can be used by reference to the examples provided herein. Other equivalent procedures can also be used. Such techniques and descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press), Hermanson, Bioconjugate Techniques, Second Edition (Academic Press, 2008); Merkus, Particle Size Measurements (Springer, 2009); Rubinstein and Colby, Polymer Physics (Oxford University Press, 2003); and the like. As utilized in accordance with teachings provided herein, the following terms, unless otherwise indicated, shall be understood to have the following meanings:

As used herein, “target sequence,” “target nucleic acid sequence,” or “target sequence of interest,” and derivatives, refers generally to any single or double-stranded nucleic acid sequence that can be amplified or synthesized according to the disclosure, including any nucleic acid sequence suspected or expected to be present in a sample. In some examples, the target sequence is present in double-stranded form and includes at least a portion of the particular nucleotide sequence to be amplified or synthesized, or its complement, prior to the addition of target-specific primers or appended adaptors. Target sequences can include the nucleic acids to which primers useful in the amplification or synthesis reaction can hybridize prior to extension by a polymerase. In some examples, the term refers to a nucleic acid sequence whose sequence identity, ordering, or location of nucleotides is determined by one or more of the methods of the disclosure.

As used herein, the term “primer” and its derivatives refer generally to any polynucleotide that can hybridize to a target sequence of interest. Typically, a primer does not include a detectable label. In some examples, the primer can also serve to prime nucleic acid synthesis. Typically, a primer functions as a substrate onto which nucleotides can be polymerized by a polymerase; in some examples, however, a primer can become incorporated into a synthesized nucleic acid strand and provide a site to which another primer can hybridize to prime synthesis of a new strand that is complementary to the synthesized nucleic acid molecule. A primer may be comprised of any combination of nucleotides or analogs thereof, which may be optionally linked to form a linear polymer of any suitable length. In some examples, a primer is a single-stranded oligonucleotide or polynucleotide. For purposes of this disclosure, the terms “polynucleotide” and “oligonucleotide” are used interchangeably herein and do not necessarily indicate any difference in length between the two. In some examples, a primer is double-stranded. If double stranded, a primer is first treated to separate its strands before being used to prepare extension products. Preferably, the primer is an oligodeoxyribonucleotide. A primer must be sufficiently long to prime the synthesis of extension products. Lengths of the primers will depend on many factors, including temperature, source of primer, and the use of the method. In some examples, a primer acts as a point of initiation for amplification or synthesis when exposed to amplification or synthesis conditions; such amplification or synthesis can occur in a template-dependent fashion and optionally results in formation of a primer extension product that is complementary to at least a portion of the target sequence. Exemplary amplification or synthesis conditions can include contacting the primer with a polynucleotide template (e.g., a template including a target sequence), nucleotides, and an inducing agent such as a polymerase at a suitable temperature and pH to induce polymerization of nucleotides onto an end of the target-specific primer. If double-stranded, the primer can optionally be treated to separate its strands before being used to prepare primer extension products. In some examples, the primer is an oligodeoxyribonucleotide or an oligoribonucleotide. In some examples, the primer can include one or more nucleotide analogs. The exact length and/or composition, including sequence, of the target-specific primer can influence many properties, including melting temperature (Tm), GC content, formation of secondary structures, repeat nucleotide motifs, length of predicted primer extension products, extent of coverage across a nucleic acid molecule of interest, number of primers present in a single amplification or synthesis reaction, presence of nucleotide analogs or modified nucleotides within the primers, and the like. In some examples, a primer can be paired with a compatible primer within an amplification or synthesis reaction to form a primer pair consisting or a forward primer and a reverse primer. In some examples, the forward primer of the primer pair includes a sequence that is substantially complementary to at least a portion of a strand of a nucleic acid molecule, and the reverse primer of the primer of the primer pair includes a sequence that is substantially identical to at least of portion of the strand. In some examples, the forward primer and the reverse primer are capable of hybridizing to opposite strands of a nucleic acid duplex. Optionally, the forward primer primes synthesis of a first nucleic acid strand, and the reverse primer primes synthesis of a second nucleic acid strand, wherein the first and second strands are substantially complementary to each other, or can hybridize to form a double-stranded nucleic acid molecule. In some examples, one end of an amplification or synthesis product is defined by the forward primer and the other end of the amplification or synthesis product is defined by the reverse primer. In some examples, where the amplification or synthesis of lengthy primer extension products is required, such as amplifying an exon, coding region, or gene, several primer pairs can be created than span the desired length to enable sufficient amplification of the region. In some examples a primer can include one or more cleavable groups. In some examples, primer lengths are in the range of about 10 to about 60 nucleotides, about 12 to about 50 nucleotides, and about 15 to about 40 nucleotides in length. Typically, a primer is capable of hybridizing to a corresponding target sequence and undergoing primer extension when exposed to amplification conditions in the presence of dNTPs and a polymerase. In some instances, the particular nucleotide sequence or a portion of the primer is known at the outset of the amplification reaction or can be determined by one or more of the methods disclosed herein. In some examples, the primer includes one or more cleavable groups at one or more locations within the primer.

As used herein, “polymerase” and its derivatives, generally refers to any enzyme that can catalyze the polymerization of nucleotides (including analogs thereof) into a nucleic acid strand. Typically, but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some examples, the second polypeptide can include a reporter enzyme or a processivity-enhancing domain. Optionally, the polymerase can possess 5′ exonuclease activity or terminal transferase activity. In some examples, the polymerase can be optionally reactivated, for example through the use of heat, chemicals or re-addition of new amounts of polymerase into a reaction mixture. In some examples, the polymerase can include a hot-start polymerase and/or an aptamer based polymerase that optionally can be reactivated.

As used herein, “amplified target sequences” and its derivatives, refers generally to a nucleic acid sequence produced by the amplification of/amplifying the target sequences using target-specific primers and the methods provided herein. The amplified target sequences may be either of the same sense (the positive strand produced in the second round and subsequent even-numbered rounds of amplification) or antisense (e.g., the negative strand produced during the first and subsequent odd-numbered rounds of amplification) with respect to the target sequences. For the purposes of this disclosure, amplified target sequences are typically less than 50% complementary to any portion of another amplified target sequence in the reaction.

As used herein, terms “ligating,” “ligation,” and derivatives refer generally to the act or process for covalently linking two or more molecules together, for example, covalently linking two or more nucleic acid molecules to each other. In some examples, ligation includes joining nicks between adjacent nucleotides of nucleic acids. In some examples, ligation includes forming a covalent bond between an end of a first and an end of a second nucleic acid molecule. In some examples, for example, wherein the nucleic acid molecules to be ligated include conventional nucleotide residues, the ligation can include forming a covalent bond between a 5′ phosphate group of one nucleic acid and a 3′ hydroxyl group of a second nucleic acid thereby forming a ligated nucleic acid molecule. In some examples, any means for joining nicks or bonding a 5′ phosphate to a 3′ hydroxyl between adjacent nucleotides can be employed. In an example, an enzyme such as a ligase can be used.

As used herein, “digestion,” “digestion step,” and its derivatives, generally refers to any process by which a cleavable group is cleaved or otherwise removed from a target-specific primer, an amplified sequence, an adaptor or a nucleic acid molecule of the sample. In some examples, the digestion step involves a chemical, thermal, photo-oxidative, or digestive process.

As used herein, the term “nucleic acid” refers to natural nucleic acids, artificial nucleic acids, analogs thereof, or combinations thereof, including polynucleotides and oligonucleotides. As used herein, the terms “polynucleotide” and “oligonucleotide” are used interchangeably and mean single-stranded and double-stranded polymers of nucleotides including, but not limited to, 2′-deoxyribonucleotides (nucleic acid) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH₄⁺, trialkylammonium, Mg₂⁺, Na⁺, and the like. An oligonucleotide can be composed entirely of deoxyribonucleotides, entirely of ribonucleotides, or chimeric mixtures thereof. Oligonucleotides can be comprised of nucleobase and sugar analogs. Polynucleotides typically range in size from a few monomeric units, e.g. 5-40, when they are more commonly frequently referred to in the art as oligonucleotides, to several thousands of monomeric nucleotide units, when they are more commonly referred to in the art as polynucleotides; for purposes of this disclosure, however, both oligonucleotides and polynucleotides may be of any suitable length. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes deoxyuridine. As discussed herein and known in the art, oligonucleotides and polynucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DINA mixture containing the desired polynucleotide of interest, followed by a precise sequence of thermal cycling in the presence of a DNA polymerase. The two primers are complementary to their respective strands of the double stranded polynucleotide of interest. To effect amplification, the mixture is denatured and the primers then annealed to their complementary sequences within the polynucleotide of interest molecule. Following annealing, the primers are extended with a polymerase to form a new pair of complementary strands. The steps of denaturation, primer annealing, and polymerase extension can be repeated many times (e.g., denaturation, annealing and extension constitute one “cycle”; there can be numerous “cycles”) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” As defined herein, target nucleic acid molecules within a sample including a plurality of target nucleic acid molecules are amplified via PCR. In a modification to the method discussed above, the target nucleic acid molecules can be PCR amplified using a plurality of different primer pairs, in some cases, one or more primer pairs per target nucleic acid molecule of interest, thereby forming a multiplex PCR reaction. Using multiplex PCR, it is possible to simultaneously amplify multiple nucleic acid molecules of interest from a sample to form amplified target sequences. It is also possible to detect the amplified target sequences by several different methodologies (e.g., quantitation with a bioanalyzer or qPCR, hybridization with a labeled probe; incorporation of biotinylated primers followed by avidin-enzyme conjugate detection; incorporation of ³²P-labeled deoxynucleotide triphosphates, such as dCTP or dATP, into the amplified target sequence). Any oligonucleotide sequence can be amplified with the appropriate set of primers, thereby allowing for the amplification of target nucleic acid molecules from genomic DNA, cDNA, formalin-fixed paraffin-embedded DNA, fine-needle biopsies and various other sources. In particular, the amplified target sequences created by a multiplex PCR process are themselves efficient substrates for subsequent PCR amplification or various downstream assays or manipulations.

As used herein “purification,” “purifying,” “purified,” or derivatives does not require absolute purity. Thus, for example, a purified nucleic acid preparation is one in which the specified nucleic acid is more enriched than the nucleic acid is in its generative environment. A preparation of substantially pure nucleic acid may be purified such that the desired nucleic acid represents at least 50% of the total nucleic acid content of the preparation. In certain examples, a substantially pure nucleic acid will represent at least 60%, at least 70%, at least 80%, at least 85%, at least 90%, at least 95%, or more of the total nucleic acid content of the preparation.

The term purified, in some examples, refers to the separation of nucleic acids of certain sizes or size ranges from a mixture of, for instance, fragmented longer nucleic acids. Thus, for instance, in one example it is appropriate to refer to purifying from a preparation of genomic DNA a collection of nucleic acid fragments of a set range of length. In each instance, the reference to a purified preparation does not require absolute purity with regard to the length of the molecules. Rather, this refers to a preparation wherein the specified nucleic acid length or range of length represents the length of at least 50% of the molecules in a mixed preparation. In certain implementations, a substantially pure nucleic acid of a specified length will contain at least 60%, at least 70%, at least 80%, at least 85%, at least 90%, at least 95%, or more nucleic acid molecules of approximately the specified length or range of length within the total nucleic acid content of the preparation.

As used herein, “variant allelic frequency” (VAF) refers to the proportion of molecules in a population of nucleic acids including the variant. In some examples, the population of nucleic acids is a cfTNA composition and the VAF refers to the proportion of a DNA variant nucleic acid fragment (such as a SNP, insertion, or deletion variant) in the composition. The VAF can be independently selected for any specific DNA variant, and may be the same as, or different from, the VAF of other DNA variants that may be present in the composition.

### II. cfTNA Compositions

The cfTNA compositions disclosed herein have a variety of uses, including but not limited to assay optimization, validation, calibration, personnel training, proficiency testing, and/or system installation. In one aspect, the present disclosure provides quality control materials with a combination of fragmented DNA and RNA variants in the background of genomic DNA (such as genomic DNA extracted from human cells). DNA variants (or mutations) include but are not limited to one or more of single nucleotide polymorphism (SNP), single nucleotide variants, substitution of one or more nucleic acids (e.g., point mutations), insertions (INS), deletions (DEL), premature stop codons, trinucleotide repeats, translocations, inversions, somatic rearrangements, allelomorphs, splice variants, regulatory variants, gene fusions, and copy number variation (CNV), for example, across several different cancer hotspot genes, while RNA variants (or mutations) include one or more of RNA fusions and isoforms (such as alternative splice forms).

Thus, in some examples, the disclosed cfTNA compositions include: a) genomic DNA comprising fragments of about 75 to about 600 bp in length; b) one or more DNA molecules comprising one or more variants selected from the group consisting of single nucleotide polymorphisms, single nucleotide variants, insertions, deletions, copy number variants, and any combination of two or more thereof; and c) one or more RNA molecules comprising one or more variants selected from fusions, isoforms, and any combination thereof.

The cfTNA compositions disclosed herein include genomic DNA (gDNA). In some examples, the compositions include fragmented gDNA, such as fragmented gDNA of a selected length or range of lengths. Thus, for instance, in some examples, the composition includes a collection of gDNA fragments of a set range of length, such as about 75 bp to about 600 bp, about 100 bp to about 500 bp, about 200 bp to about 400 bp, or about 150 bp to about 250 bp. In one specific example, the collection of gDNA fragments ranges from about 180 bp to about 220 bp, for example, having a peak at about 200 bp. In particular examples, gDNA may be obtained, for example, from one or more cells by methods known to those of ordinary skill in the art (for example, kits for this purpose are commercially available). In other examples, gDNA may be commercially available. In some examples, the gDNA is eukaryotic gDNA, for example human gDNA. In one example, the gDNA is isolated from a reference cell line, such as from NIST Genome in a Bottle. In a specific example, the gDNA is isolated from GM24385 reference cell line. The gDNA is fragmented, purified, and included in the cfTNA composition as described herein.

The cfTNA compositions disclosed herein also include one or more DNA variants (such as 1, 2, 3, 4, 5, 6, 7, 8, 9, 10, 11, 12, 13, 14, 15, 16, 17, 18, 19, 20, or more DNA variants). In other examples, the cfTNA compositions include 20 or more, 100 or more, 200 or more, or even 500 or more DNA variants. In some examples, the composition includes one or more DNAs, including one or more plasmids, artificial chromosomes, and/or double-stranded DNA fragments (such as gBlocks) including one or more DNA variants. The variants may be variants from a single gene or more than one gene (such as 1, 2, 3, 4, 5, 6, 7, 8, 9, 10, 11, 12, 13, 14, 15, 16, 17, 18, 19, 20, 50, 100, 200, 500, or more genes). The DNA variants may be selected from one or more of single nucleotide polymorphism (SNP), single nucleotide variants, substitution of one or more nucleic acids (e.g., point mutations), insertions (INS), deletions (DEL), premature stop codons, trinucleotide repeats, translocations, inversions, somatic rearrangements, allelomorphs, splice variants, regulatory variants, gene fusions, copy number variation (CNV), and any combination thereof. In some examples, the DNA variants may be selected from one or more of single nucleotide polymorphism (SNP), insertion (INS), deletion (DEL), inversions, duplication, substitution, copy number variation (CNV), translocation, gene rearrangement, alternative splicing, gene fusion, and any combination of two or more thereof. In some examples, the composition includes at least one SNP, at least one insertion, at least one deletion, at least one splice variant, and at least one copy number variant fragment.

In some examples, the DNA molecules including one or more variants are included in the composition in the form of plasmid DNA or artificial chromosome DNA (such as human artificial chromosome (HAC), yeast artificial chromosome (YAC), bacterial artificial chromosome (BAC), P1-derived artificial chromosome (PAC)), cosmid, or fosmid DNA including the DNA variant(s)). In some examples, the DNA including the variant(s) (such as the plasmid or artificial chromosome) is fragmented, purified, and included in the cfTNA composition as described herein. In some examples, the fragmented DNA is of a selected length or range of lengths. Thus, in some examples, the composition includes a collection of DNA fragments (such as plasmid or artificial chromosome DNA fragments) of a set range of length, such as about 100 bp to about 500 bp, for example, about 100 bp to about 200 bp, about 150 bp to about 250 bp, about 200 bp to about 300 bp, about 250 bp to about 350 bp, about 300 bp to about 400 bp, about 350 bp to about 450 bp, or about 400 to 500 bp. In one specific example, the collection of DNA variant fragments ranges from about 100 bp to about 500 bp, having a peak at about 240 bp. In other examples, the DNA variant fragments are from gBlock DNA fragments including one or more DNA variants. In some examples, the gBlock DNA fragments are about 100 bp to about 500 bp, for example, about 100 bp to about 200 bp, about 150 bp to about 250 bp, about 200 bp to about 300 bp, about 250 bp to about 350 bp, about 300 bp to about 400 bp, about 350 bp to about 450 bp, or about 400 to 500 bp, for example, about 200 bp.

The cfTNA compositions disclosed herein also include one or more (such as 1, 2, 3, 4, 5, 6, 7, 8, 9, 10, 11, 12, 13, 14, 15, 16, 17, 18, 19, 20, or more) RNA molecules, such as RNA variants. In other examples, the cfTNA compositions include 20 or more, 100 or more, 200 or more, or even 500 or more RNA variants. The RNA variants may be selected from RNA fusions, isoforms (e.g., splice variants), and any combination thereof. In some examples, the composition includes at least one RNA fusion and at least one isoform. In some examples, the one or more RNA variants are generated in vitro, for example by in vitro transcription from a DNA, such as plasmid DNA.

The cfTNA compositions provided herein include one or more DNA variants (such as one or more DNA fragments including a variant) and one or more RNA variants (such as one or more RNA molecules including a variant) in a gDNA fragment mixture. In some examples, the composition includes purified gDNA fragments at a concentration of about 1-1000 ng/μL (such as about 1-100 ng/μL, about 50-150 ng/μL, about 125-200 ng/μL, about 175-300 ng/μL, about 250-400 ng/μL, about 300-500 ng/μL, about 450-600 ng/μL, about 550-700 ng/μL, about 650-800 ng/μL, about 750-900 ng/μL, or about 850-1000 ng/μL). In one example, the composition includes purified gDNA fragments at a concentration of about 40 ng/u L to about 60 ng/μL. The one or more DNA variants and one or more RNA variants are present with the gDNA fragments in a defined amount.

Thus, in some examples, each of the one or more SNP, SNV, nucleic acid substitution, insertion, or deletion DNA variants are present in the composition at a defined variant allelic frequency (VAF). In some examples, the VAF of each of the DNA variants in the composition is about 0.1% to about 50% (for example about 0.1% to about 5%, about 1% to about 10%, about 2.5% to about 15%, about 7.5% to about 25%, about 20% to about 35%, about 30% to about 45%, or about 40% to about 50%. For example, the VAF of each of the DNA variants is about 0.1%, about 0.2%, about 0.3%, about 0.4%, about 0.5%, about 0.6%, about 0.7%, about 0.75%, about 0.8%, about 0.9%, about 1%, about 1.1%, about 1.2%, about 1.3%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. The VAF of each of the one or more DNA variants in the composition may be independently selected and may be the same as or different from the VAF of other DNA variants in the composition. In some examples, the variant allelic frequency of each of the one or more SNP, insertion, or deletion DNA variants in the composition may independently vary by up to about +30% from the defined amount.

In additional examples, each of the one or more CNV DNAs are present in the composition at a defined copy number. Thus, in some examples, the copy number of each of the CNV DNAs present in the composition is about 2 to about 60 copies (for example, about 2 to about 10, about 5 to about 15, about 7.5 to about 20, about 12 to about 25, about 20 to about 30, about 25 to about 40, about 35 to about 50, or about 45 to about 60 copies). For example, the number of copies of each of the CNV DNAs is about 2, about 2.3, about 2.5, about 3, about 3.5, about 4, about 4.5, about 5, about 5.5, about 6, about 6.5, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40 about 41, about 42, about 43, about 44, about 45, about 46, about 47, about 48, about 49, about 50, about 51, about 52, about 53, about 54, about 55, about 56, about 57, about 58, about 59, or about 60 copies. In one example, each of the one or more CNV DNA nucleic acids are present at about 5.6 copies. The copy number of each of the one or more CNV DNAs in the composition may be independently selected and may be the same as or different from the copy number of other CNV DNAs in the composition. In some examples, the copy number of each of the CNV DNAs may independently vary by up to about ±20% from the defined amount.

In further examples, each of the RNA variants (e.g., fusion or isoform variants) are present in the composition at a defined copy number. In some examples, the copy number of each of the RNA variants present in the composition is about 25-125 copies (such as about 25, about 30, about 35, about 40, about 45, about 50, about 55, about 60, about 65, about 70, about 75, about 80, about 85, about 90, about 95, about 100, about 105, about 110, about 115, about 120, or about 125 copies). The copy number of each of the one or more RNA variants in the composition may be independently selected and may be the same as or different from the copy number of other RNA variants in the composition. In some examples, the copy number of each of the RNA variants may independently vary by up to about +25% from the defined amount.

The disclosed cfTNA compositions may be formulated in a suitable aqueous solution, such as a sterile aqueous solution. In some examples, the compositions disclosed herein are formulated in a solution that is compatible with an assay in which the composition will be used as a control. Thus, in some examples, the cfTNA composition is formulated in a solution that is compatible with next-generation (e.g., massively parallel) sequencing technologies, such as pyrosequencing (for example, as commercialized by 454 Life Sciences, Inc., Branford, Conn.); sequencing by ligation (for example, as commercialized in the SOLID™. technology, Life Technologies, Inc., Carlsbad, Calif.); sequencing by synthesis using modified nucleotides (such as commercialized in TruSeq™ and HiSeg™ and MiSeq™ and/or NovaSeq™ technology by Illumina, Inc., San Diego, Calif.; HeliScope™ by Helicos Biosciences Corporation, Cambridge, Mass.; and PacBio Sequel® or RS systems by Pacific Biosciences of California, Inc., Menlo Park, Calif.), sequencing by ion detection technologies (e.g., Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and similar highly parallelized sequencing methods. In some examples, the aqueous solution does not include Tris (e.g., tris(hydroxymethyl)aminomethane). In other examples, the cfTNA composition is in a dry format, e.g., a lyophilized form of the composition. In other examples, the cfTNA composition may be spiked into plasma (such as human plasma) or synthetic plasma.

In some examples, the cfTNA composition includes one or more DNA variants and/or one or more RNA variants with sequences including mutations or variants associated with cancer or other diseases or disorders (including genetic disorders). In some examples, the composition includes one or more DNA variant and/or one or more RNA variant having mutations or variants associated with one or more solid tumor cancers selected from the group consisting of head and neck cancers (e.g., HNSCC, nasopharyngeal, salivary gland), brain cancer (e.g., glioblastoma, glioma, gliosarcoma, glioblastoma multiforme, neuroblastoma), breast cancer (e.g., TNBC, trastuzumab resistant HER2+ breast cancer, ER+/HER− breast cancer), gynecological (e.g., uterine, ovarian cancer, cervical cancer, endometrial cancer, fallopian cancer), colorectal cancer, gallbladder cancer, esophageal cancer, gastrointestinal cancer, gastric cancer, bladder cancer, prostate cancer, testicular cancer, urothelial cancer, liver cancer (e.g., hepatocellular carcinoma, HCC), lung cancer (e.g., non-small cell lung cancer, small cell lung cancer), kidney (renal cell) cancer, pancreatic cancer (e.g., adenocarcinoma, ductal), thyroid cancer, bile duct cancer, pituitary tumor, Wilms' tumor, Kaposi sarcoma, hairy cell carcinoma, osteosarcoma, thymus cancer, skin cancer, melanoma, heart cancer, oral and larynx cancer, neuroblastoma, mesothelioma, and other solid tumors (e.g., thymic, bone, soft tissue, oral SCC, myelofibrosis, synovial sarcoma). In other examples, the composition includes one or more DNA variant and/or one or more RNA variant having mutations or variants associated with one or more blood/hematologic cancers selected from the group consisting of multiple myeloma, diffuse large B cell lymphoma (DLBCL), lymphoma, Hodgkins lymphoma, non-Hodgkins lymphoma, follicular lymphoma, leukemia, acute myeloid leukemia (AML), chronic lymphocytic leukemia (CLL), and myelodysplastic syndrome. In some examples, the cfTNA composition includes one or more DNA variants and one or more RNA variants including sequences having mutations or variants in targets selected from those listed in Table 1.

TABLE 1
Exemplary targets by variant class

| | | | Inter-Genetic | Intra-Genetic |
|---|---|---|---|---|
| DNA Variants | | CNV | Fusions | Fusions |
| AKT1 | HRAS | ALK | ALK | AR |
| AKT2 | IDH1 | AR | BRAF | EGFR |
| AKT3 | IDH2 | CD274 | ESR1 | MET |
| ALK | KEAP1 | CDKN2A | FGFR1 | |
| AR | KIT | EGFR | FGFR2 | |
| ARAF | KRAS | ERBB2 | FGFR3 | |
| BRAF | MAP2K1 | ERBB3 | MET | |
| CDK4 | MAP2K2 | FGFR1 | NRG1 | |
| CHEK2 | MET | FGFR2 | NTRK1 | |
| CTNNB1 | MTOR | FGFR3 | NTRK2 | |
| EGFR | NRAS | KRAS | NTRK3 | |
| ERBB2 | NTRK1 | MET | NUTM1 | |
| ERBB3 | NTRK2 | PIK3CA | RET | |
| ERBB4 | NTRK3 | PTEN | ROS1 | |
| ESR1 | PDGFRA | | RSPO2 | |
| FGFR1 | PIK3CA | | RSPO3 | |
| FGFR2 | PTEN | | | |
| FGFR3 | RAF1 | | | |
| FGFR4 | RET (Exon 12) | | | |
| FLT3 | RET | | | |
| GNA11 | STK11 | | | |
| GNAQ | ROS1 | | | |
| GNAS | SMO | | | |
| | TP53 | | | |

In some examples, the cfTNA composition includes one or more DNA fragments including variants (such as one or more SNPs, insertions, and/or deletions) in one or more (such as 1, 2, 3, 4, 5, 6, 7, 8, or all) of EGFR, KRAS, KEAP1, BRAF, KIT, STK11, RET, ERBB2, and MET. In other examples, the cfTNA composition includes one or more DNA fragments including CNVs in ERBB2, MET, or both. In additional examples, the cfTNA composition includes one or more RNAs including a fusion selected from one or more (such as 1, 2, 3, 4, 5, or all) of EMLA-ALK, TPM3-NTRK1, NACC2-NTRK2, ETV6-NTRK3, CCDC6-RET, and SLC34A2-ROS1. In another example, the cfTNA compositions includes an RNA including an alternative splice form of MET.

In a specific example, the cfTNA composition includes nucleic acids including one or more of the variants, fusions, and isoforms listed in Table 6. In one examples, the composition includes nucleic acids including each of the variants, fusions, and isoforms listed in Table 6.

### III. Methods of Preparing cfTNA Compositions

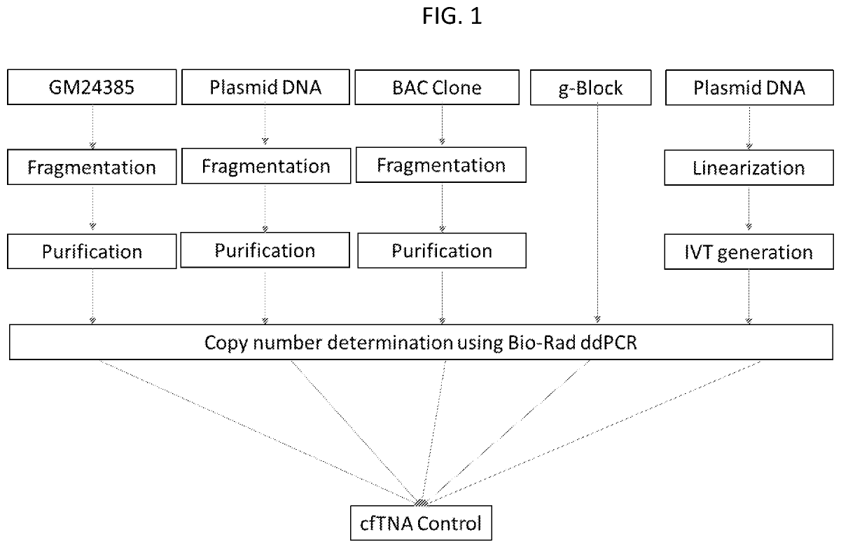

Methods of preparing cfTNA compositions (such as those described in Section II) are provided herein. In some examples, the methods include combining: a) a population of genomic DNA fragments; b) one or more DNA molecules comprising one or more variants selected from the group consisting of single nucleotide polymorphisms, single nucleotide variants, insertions, deletions, copy number variants, and any combination of two or more thereof; and c) one or more RNA molecules comprising one or more variants selected from fusions, isoforms, and any combination thereof. An exemplary work flow for preparing a disclosed cfTNA composition is provided as FIG. 1.

In some examples, the population of gDNA fragments included in the composition is prepared by fragmenting gDNA to a selected length or range of lengths, such as about 75 bp to about 600 bp, about 100 bp to about 500 bp, about 200 bp to about 400 bp, or about 150 bp to about 250 bp. In one specific example, the selected length ranges from about 180 bp to about 220 bp, for example, having a peak at about 200 bp. The fragmented gDNA may be purified from other components in the preparation, such as other cellular components. As noted above, purification does not require absolute purity. In particular examples, the purified gDNA fragment preparation may include small amounts of RNA (e.g., total RNA). In some examples, the gDNA preparation includes up to about 10% total RNA (such as about 10%, about 9%, about 8%, about 7%, about 6%, about 5%, about 4%, about 3%, about 2%, or about 1% total RNA). In some examples, the gDNA preparation includes about 10% total RNA. In particular examples, total RNA is added to the gDNA preparation to achieve the desired amount of total RNA in the preparation.

Any suitable method of purification can be used to obtain the purified gDNA fragments. In one non-limiting example, the gDNA fragments are purified using a magnetic bead-based method (such as AMPure XP beads, Beckman Coulter). In some examples, the purified gDNA is in a suitable buffer. The concentration of the purified gDNA fragment preparation may be determined by any suitable method, such as fluorometric or spectrophotometric methods. In one example, the concentration of the purified gDNA fragments is determined using Qubit DNA HS assay (Invitrogen) or NanoDrop nucleic acid quantification (Thermo Fisher). The size of the fragments in the preparation may also be determined by any suitable method. In some examples, the size of the gDNA fragments is determined using the TapeStation platform or BioAnalyzer system (both Agilent).

In some examples, the one or more DNA molecules with one or more DNA variants included in the composition are prepared by fragmenting DNA including one or more variants (such as plasmid or BAC DNA including one or more variants) to a selected length or range of lengths, such as about 100 bp to about 500 bp, for example, about 100 bp to about 200 bp, about 150 bp to about 250 bp, about 200 bp to about 300 bp, about 250 bp to about 350 bp, about 300 bp to about 400 bp, about 350 bp to about 450 bp, or about 400 to 500 bp. In one specific example, the DNA is fragmented to a range of about 100 bp to about 500 bp, having a peak at about 240 bp. The fragmented DNA may be purified from other components in the preparations. As noted above, purification does not require absolute purity. Any suitable method of purification can be used to obtain the purified DNA fragments. In one non-limiting example, the DNA fragments are purified using a magnetic bead-based methods (such as AMPure XP beads, Beckman Coulter). In some examples, the purified DNA is in a suitable buffer, such as TE buffer. In other examples, DNA including one or more variants (such as plasmid DNA, BAC DNA, and/or gBlock DNA) is not fragmented prior to inclusion in the final composition. The concentration of the DNA preparation may be determined by any suitable method, such as fluorometric or spectrophotometric methods. In one example, the concentration of the DNA is determined using Qubit DNA HS assay (Invitrogen) or NanoDrop nucleic acid quantification (Thermo Fisher). The size of fragments in the preparation may also be determined by any suitable method. In some examples, the size of the DNA fragments is determined using the TapeStation platform or BioAnalyzer system (both Agilent).

In some examples, the one or more RNA molecules included in the composition are prepared by in vitro transcription (IVT) of corresponding DNA(s) encoding the one or more RNAs. The DNA may be included in a plasmid and the methods may include linearizing the plasmid prior to IVT. Plasmid DNA can be linearized by any technique, including endonuclease digestion (e.g., restriction enzyme digestion). In other examples, the DNA may be in the form of a double-stranded DNA fragment (such as a gBlock DNA fragment), and may not require additional processing prior to IVT. In some examples, the concentration of the linearized plasmid or double-stranded DNA fragment is determined prior to IVT. Any method of IVT can be used to produce the RNA, for example using T7 RNA polymerase. For example, commercial kits for IVT are available from many sources. Following IVT, the RNA transcript can be diluted (for example, to a desired concentration) in a suitable aqueous solution. The concentration of the RNA may be determined by any suitable method, such as fluorometric or spectrophotometric methods. In one example, the concentration of the RNA is determined using Qubit RNA HS assay (Invitrogen). The size and purity of the RNA may also be determined by any suitable method. In some examples, the size of the RNA is determined using High Sensitivity RNA ScreenTape Assay (Agilent).

The method also includes determining the copy number of each of the gDNA, DNA molecules, and RNA molecules to be included in the cfTNA composition. Any suitable method can be utilized. In some examples, the copy number is determined using droplet digital PCR (ddPCR). In some examples, the DNA molecules from plasmid, artificial chromosome, and/or double-stranded DNA fragments (such as gBlocks) are mixed with gDNA prior to ddPCR. In other examples, the RNA molecules are mixed with gDNA prior to ddPCR. Exemplary methods are provided in Example 1; however, other methods can be utilized to determine copy number, such as digital PCR, or NGS.

The method further includes mixing or combining the gDNA, DNA fragments, and RNAs to arrive at the cfTNA composition. The components are combined in selected amounts, such as those described in Section II. In some examples, the DNA and RNA components are spiked into the fragmented, purified gDNA. The final composition may be formulated in an appropriate aqueous solution. In some examples, the aqueous solution is Nucleic Acid Dilution Solution as matrix. In some examples, the aqueous solution does not include Tris (e.g., tris(hydroxymethyl)aminomethane). In other examples, the cfTNA composition is further mixed with plasma, such as human plasma. In some examples, the plasma is DNA depleted human plasma.

In some examples, the provided methods include fragmenting nucleic acids (such as gDNA, plasmid DNA, and/or artificial chromosome (e.g., BAC) DNA), such as with any one or any combination of mechanical stress, changes in temperature or pH, chemical compounds, enzymatic reactions (such as endonucleases or restriction enzymes), and/or radiation energy. In some examples, mechanical stresses include shearing forces, fluid shear, hydrodynamic shear, pulsatile shear, or cavitation. In some examples, mechanical forces that can damage nucleic acids include acoustic force, nebulizing force, sonication force, cavitation force, or shearing force. In some examples, apparatuses that can be used to fragment nucleic acids include acoustic shearing apparatuses, nebulizing apparatuses, sonication apparatuses, needle shearing apparatuses, or French press apparatuses. Other examples of mechanical stress that can fragment nucleic acids include pipetting, stirring, centrifuging, pumping, filtering, spray drying, injecting, or container-filling. The particular method and fragmentation conditions can be selected to obtain fragments of a desired size or range of sizes. Exemplary methods and conditions are provided in Example 1; however, a person of ordinary skill in the art can select alternative methods and conditions.

### IV. Kits

Also provided are kits including one or more of the cfTNA compositions disclosed herein. The kit may also include a container (e.g., a vial, test tube, flask, bottle, syringe, or other packaging system) into which the one or more cfTNA compositions may be placed/contained. Where more than one component is included in the kit, it will generally include at least one second, third, or other additional container(s) into which the additional components can be separately placed. Various combinations of components may also be packaged in a single container. The kits may also include reagent containers in close confinement for commercial sale. The kit may include the cfTNA composition in a liquid solution (such as an aqueous solution) or in a dry form (e.g., lyophilized) that can be reconstituted by a user. In other examples, the cfTNA composition is incorporated into FFPE blocks.

In some examples, the kit is for use in methods of detecting one or more biomarkers, such as one or more biomarkers relating to a disease or disorder (e.g., cancer, genetic disorders, or infectious disease) using next-generation sequencing or other methods (such as digital PCR or qPCR). In one example, the kit is for use in methods of detecting one or more biomarkers associated with cancer (including one or more SNPs, mutations, CNVs, and fusions). In some examples, the kit includes a cfTNA composition that includes one or more (or each) of the biomarkers detected in the method.

The kit may also include instructions for employing the kit components as well as the use of any other reagent not included in the kit. Instructions may include variations that may optionally be implemented. The instructions may be provided as a separate part of the kit (e.g., a paper or plastic insert or attachment) or as an internet-based application. Kits may further include one or more of a polymerase, one or more oligonucleotide primers, one or more buffers, other reaction mixtures, or any combination thereof. Other variations and arrangements for the kits of this disclosure are contemplated as would be understood by those of ordinary skill in the art.

### V. Methods of Use

This disclosure also provides methods for confirming the validity of an assay (such as a next-generation sequencing assay) by including a cfTNA composition disclosed herein in a sample. In some examples, the cfTNA composition includes a known number of representative sequences and/or variants thereof and detection of all of the representative sequences and/or variants in the mixture indicates the sequencing reaction was accurate.

The cfTNA control can be used during NGS method development, method validation, operator training, and/or routine QC monitoring. The control is fragmented and can closely mimic a patient sample. During assay development, the NOS assay may be developed using the cfTNA control to align the VAP of specific variants. For validation purposes, the cfTNA control can be used to test the assay accuracy, precision, repeatability and reproducibility. The control can also be used for new operator training, lab proficiency testing, and routine testing to monitor any systematic errors.

### EXAMPLES

The following examples are provided to illustrate certain particular features and/or examples. These examples should not be construed to limit the disclosure to the particular features or examples described.

### Example 1

Preparation of a cfTNA Control

### Fragmentation of Genomic DNA

Background human genomic DNA (gDNA) was extracted from the NIST Genome in a Bottle (GM24385) reference cell line. Up to 50 μg of gDNA was formulated with fragmentation buffer containing 1× TE buffer (Ambion AM9849), 1 mM 3-Indolepropionic acid, 1 mM EDTA at a total 130 μL volume. The mixture was transferred to an AFA Fiber snap-Cap tube (Covaris catalog. 520217) and fragmented using Covaris ME220 focused-ultrasonicator. The fragmentation program is listed in Table 2. The gDNA may contain certain amount of total RNA, for certain application purposes, e.g. NGS normalization.

TABLE 2
gDNA fragmentation conditions
8 microTUBE-130 AFA Fiber snap-Cap (PN 520217)
Rack 8 microTUBE-130 Strip v2 (PN520217.2)

| | |
|---|---|
| Sample Volume (μL) | 130 μL ± 5 μL |
| gDNA input (μg) | up to 50 μg |
| Buffer | TE—Tris-EDTA, pH 8.0 |
| Temperature (° C.) | Set: 12° C. (Range: 7-15° C.) |

| Parameters used for expected size | Condition 15 |
|---|---|
| Expected Size (bp) | 200 bp |
| Duration (s) | 10 |
| Peak Power (W) | 70 |
| Duty Factor (%) | 20 |
| Cycles per Burst (#) | 1000 |
| Delay (s) | 5 |
| Total cycle# | 30 |

After fragmentation, the gDNA was pooled and purified using AMPure XP beads (Beckman Coulter A63881) at 1.6× volume of the total fragmented gDNA. The AMPure XP beads were warmed up to room temperature for 30 mins before adding to the fragmented gDNA. The beads-DNA mixture was incubated for 10 mins at room temperature, and the bead-DNA fraction was separated from the solution by placing the sample tubes on a magnetic rack for 5 mins. After removing the supernatant and washing twice with 500 μL freshly prepared 80% ethanol, the bead-DNA fraction was air-dried for 5 mins. The fragmented gDNA was then eluted from the beads by adding 35 μL TE buffer.

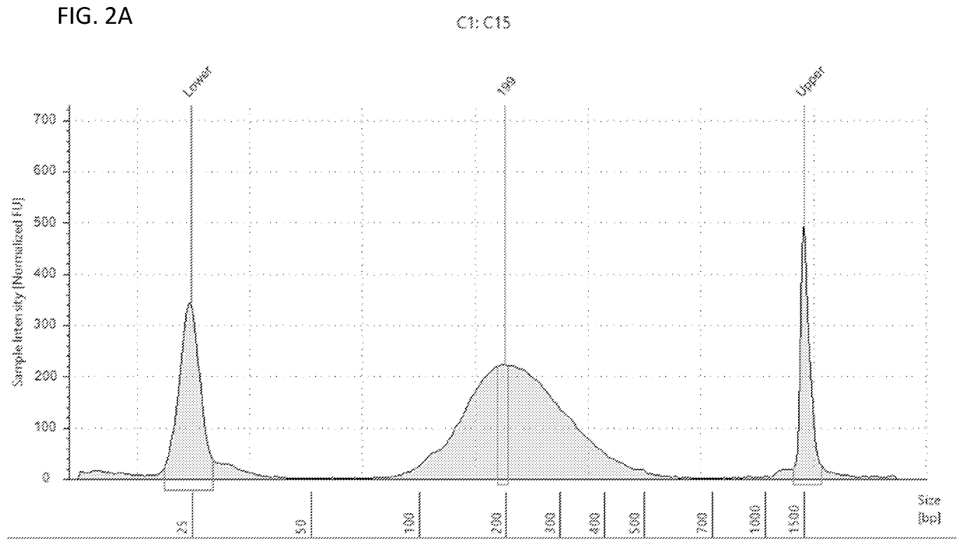

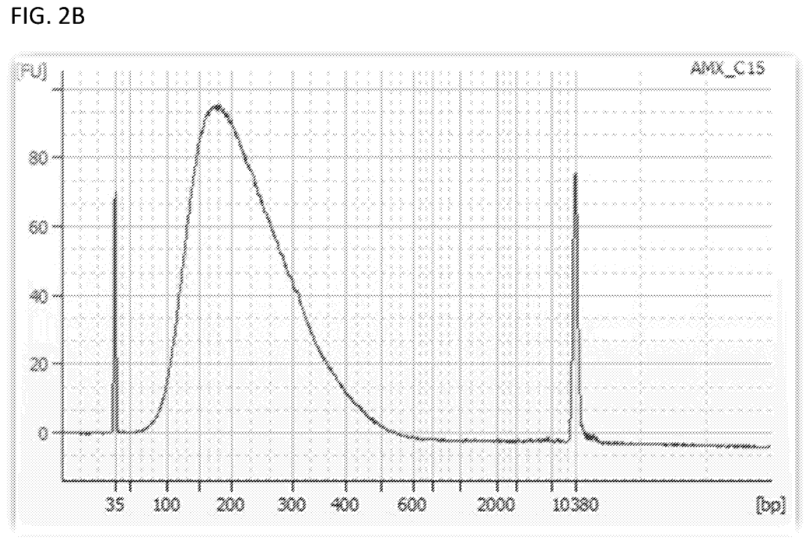

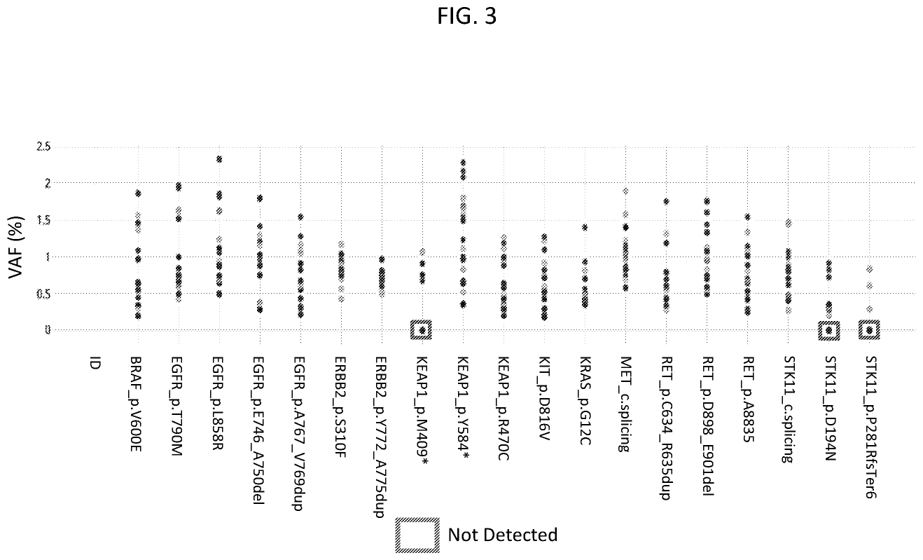

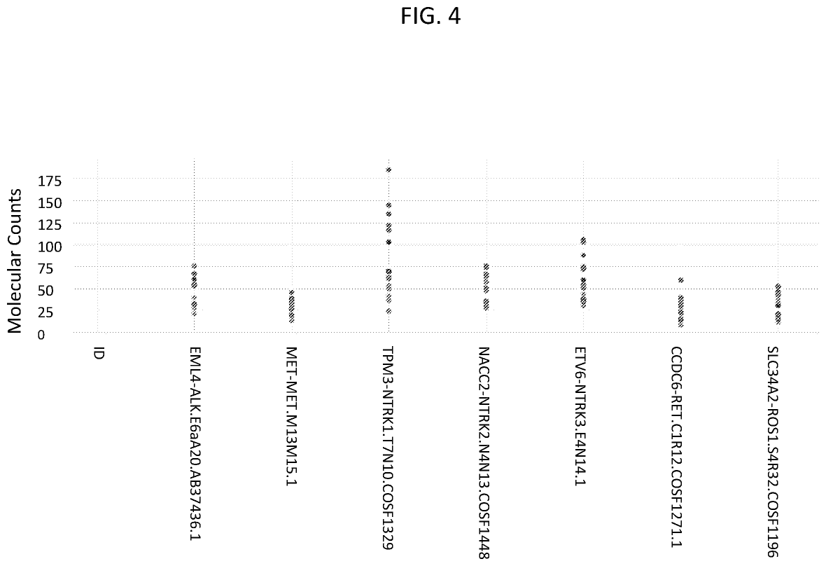

The size of the purified fragmented gDNA was determined using High Sensitivity D1000 ScreenTape assay and reagents (Agilent 5067-5584) on Agilent 2200 TapeStation system. The concentration of the gDNA was measured by Qubit DNA HS assay kit (Invitrogen Q32951) using Qubit 3.0 fluorometer, diluted down to 1 ng/μL. Then, 2 μL of the diluted gDNA was mixed with 2 μL of the High Sensitivity D1000 sample buffer, mixed at 2000 rpm for 1 min followed by quick spin before loading on to the ScreenTape system. Expected fragmented gDNA size ranged from 75-600 bp with a peak size at 200 bp (+20 bp) (FIGS. 2A and 2B).

### Design of the Synthetic Mutation Constructs

The design of several DNA synthetic mutation constructs in the cfTNA control utilized the MegaMix technology described in U.S. Pat. No. 10,364,465. The DNA fragments started with unique Xeno sequence followed by 1-6 DNA fragments, each of which has mutation at the center of the fragment flanked by 120-150 bp wild-type sequences (Table 3). The DNA fragments can be in the form of linearized plasmid (Genscript custom order) or gBlock™ fragments (Integrated DNA Technologies). To create copy number variation, purified Bacteria Artificial Chromosome (BAC clone) that carries specific large human gene (e.g., MET or ERBB2 gene fragments; Table 4) was used.

TABLE 3
DNA constructs

| | | | | Onco |
|---|---|---|---|---|
| | Variant | Most Prevalent | | DNA |
| Gene | Type | Variant ID | Protein Change | Construct |
| Xeno 1 | | | | 1 |
| EGFR | SNP | COSM6240 | T790M | |
| EGFR | INS | COSM12376 | A767_V769dup | |
| EGFR | SNP | COSM6224 | L858R | |
| EGFR | DEL | COSM6223 | E746_A750del | |
| KEAP1 | INS | COSM6201646 | Y584* | |
| KRAS | SNP | COSM516 | G12C | |
| Xeno 2 | | | | 2 |
| STK11 | DEL | COSM20871 | P281RfsTer6 | |
| KEAP1 | SNP | COSM4564847 | R470C | |
| BRAF | SNP | COSM476 | V600E | |
| KIT | SNP | COSM1314 | D816V | |
| KEAP1 | DEL | COSM6916353 | M409* | |
| Xeno 3 | | | | 3 |
| RET | INS | C634_R635dup | N/A | |
| RET | DEL | COSM962 | D898_E901del | |
| Xeno 12 | | | | 12 |
| ERBB2 | SNP | COSM48358 | S310F | |
| ERBB2 | INS | COSM12558 | Y772_A775dup | |
| Xeno 13 | | | | 13 |
| MET | SNV | COSM6108462 | c.3082 + 1G > T | |
| Xeno 15 | | | | 15 |
| STK11 | SNP | COSM25847 | D194N | |
| Xeno 16 | | | | 16 |
| RET | SNP | COSM133167 | p.A883S | |
| STK11 | DEL | OMINDEL1012 | c.598- | |
| | | | 2_600delAGGCA | |

TABLE 4
BAC clones

| BAC clone Name | Clone ID | Chromosome location |
|---|---|---|
| ERBB2 | CTD-2019C10 | chr17: 39577926-39786803 |
| MET | CTD-2270N20 | Chr7: 116297432-116459846 |

### Generation of In Vitro Transcription RNA

Synthetic RNA transcripts containing fusion isoform or imbalance copy genes (Table 5) were generated using either DNA plasmids or gBlock DNA fragments (Genscript), and in vitro transcribed using MEGAscript T7 transcription kit (Invitrogen AM1333). The concentration of the linearized plasmids or gBlock™ DNA fragments were determined by Qubit DNA HS assay kit (Invitrogen Q32851), and a total of 1 μg of DNA template was added into 20 μL in vitro transcription reaction mixtures containing 7.5 mM of ATP, CTP, GTP and UTP respectively, 1× reaction buffer, 2 μL of RNA polymerase mix. The mixture was incubated at 37° C. for 4 hours or overnight in a Veriti Dx thermal cycler (Invitrogen). The reaction was terminated by 1 μL of TURBO DNase at 37° C. for 15 mins followed by purification steps using MEGAclear kit (Invitrogen AM1908). The RNA transcript was eluted with 50 μL of pre-heated elution solution. The purified IVT RNA was then diluted by 20-fold (1:20) and the concentration was determined by Qubit RNA HS assay kit (Invitrogen Q32855). The size and the purity of the IVT RNA was determined by High Sensitivity RNA ScreenTape assay (Agilent 5067-5579) on Agilent 2200 TapeStation system.

TABLE 5
IVT RNA Constructs

| IVT RNA | Type | Variant/Fusion Isoform |
|---|---|---|
| Onco RNA 6 | Fusion | NACC2-NTRK2.N4N13.COSF1448 |
| Onco RNA 8 | Fusion | EML4-ALK.E6aA20.AB374361.1 |
| Onco RNA 9 | Fusion | CCDC6-RET.C1R12.COSF1271.1 |
| Onco RNA 10 | Fusion | TPM3-NTRK1.T7N10.COSF1329 |
| Onco RNA 12 | Fusion | ETV6-NTRK3.E4N14.1 |
| Onco RNA 15 | Fusion | MET-MET.M13M15.1 |
| Onco RNA 16 | Fusion | SLC34A2-ROS1.S4R32.COSF1196 |

### Copy Number Determination of DNA and RNA

The copy number of gDNA, BAC clones, synthetic DNA, and IVT RNA targets for the cfTNA reference materials were all determined by QX200 Bio-Rad Droplet Digital PCR (ddPCR) system (Bio-Rad 1864001). For DNA targets (BAC clones, plasmid or gBlock™), the concentration of each target was determined by Qubit DNA HS assay kit (Invitrogen Q32951), and serially diluted down to 1000 cp/u L using TE buffer (Ambion AM9849) by estimation of the copy number based on the construct size using the calculation below. The fragmented gDNA was diluted 40-fold using TE buffer for copy number determination using the ddPCR system.

$$Number of copies = ( ng * number / mole ) / ( bp * ng / g * g / mole of bp ) or Number of copies / μL = ( concentration * 6.022 \times 1023 ) / ( length * 1 \times 10 \hat{} 9 * 650 )$$

$$Where concentration = concentration obtained by Qubit assay ( nominal concentration )$$

For RNA targets the concentration of the each target was determined by Qubit RNA HS assay kit (Invitrogen Q32855), and serially diluted down to 5000 cp/μL using NADS (nucleic acid dilution solution) by estimation of the copy number based on the construct size using the calculation below.

$$Number of copies = ( ng * number / mole ) / ( bp * ng / g * g / mole of bp ) or Number of copies / μL = ( concentration * 6.022 \times 1023 ) / ( length * 1 \times 10 \hat{} 9 * 330 )$$

$$Where concentration = Concentration obtained by Qubit assay ( nominal concentration )$$

Up to 4.8 μL of diluted DNA at 1000 cp/μL and diluted gDNA was added into 24 μL of the ddPCR reaction, which also included ddPCR 2× Supermix for probe (no dUTP, Bio-Rad 186-3024). Up to 2.4 μL of the diluted RNA at 5000 cp/μL was added into 24 μL of the ddPCR reaction, which also include One-Step RT-ddPCR Advance Kit for Probes (Bio-Rad 1864021 or 1864022).

The droplets were generated using QX200 Droplet Generator and loaded onto a 96-well PCR plate (semi-skirted blue, Eppendorf 951020362) before putting on the Veriti Dx 96-Well PCR Thermal Cycler (Applied Biosystems, 4452300).

The cycling conditions were as follows for DNA:

| Steps | Hold 1 | 40 Cycles | | Hold 2 | Hold 3 |
|---|---|---|---|---|---|
| Temperature | 95 | 94 | 60 | 98 | 4 |
| (° C.) | | | | | |
| Time | 10 min | 30 sec | 60 sec | 10 min | Infinite |

The cycling conditions were as follows for RNA:

| Steps | Hold 1 | Hold 2 | 40 Cycles | | Hold 3 | Hold 4 |
|---|---|---|---|---|---|---|
| Temperature | 50 | 95 | 95 | 60 | 98 | 4 |
| (° C.) | | | | | | |
| Time | 60 min | 10 min | 30 sec | 60 sec | 10 min | Infinite |

The plate was then moved to QX200 Droplet Reader and analysed using QuantSoft Version 1.7.4 software.
Manufacturing of cfTNA Control

The cfTNA control was manufactured by combining the synthetic plasmids, gBlocks, BAC clones and IVT RNA at target concentration based on ddPCR copy number results, and spiking into the fragmented, purified, gDNA at 40-60 ng/μL final concentration. The product was formulated in NADS (nucleic acid dilution solution) as matrix. The final target specification is listed in Table 6.

TABLE 6
Specification for cfTNA Controls

| | | | | | Specification |
|---|---|---|---|---|---|
| | | | | | Variant Allelic |
| Gene | Variant | | DNA Variant/Fusion | DNA/ | Frequency (VAF)/ |
| Name | Type | VARIANT ID | Isoform | RNA | Copies |
| EGFR | SNP | COSM6240 | T790M | DNA | 1.00% +/− 0.30 |
| EGFR | SNP | COSM6224 | L858R | DNA | 1.00% +/− 0.30 |
| EGFR | INS | COSM12376 | A767_V769dup | DNA | 1.00% +/− 0.30 |
| EGFR | DEL | COSM6223 | E746_A750del | DNA | 1.00% +/− 0.30 |
| KRAS | SNP | COSM516 | G12C | DNA | 1.00% +/− 0.30 |
| KEAP1 | INS | COSM6201646 | Y584* | DNA | 1.00% +/− 0.30 |
| BRAF | SNP | COSM476 | V600E | DNA | 0.90% +/− 0.27 |
| KIT | SNP | COSM1314 | D816V | DNA | 0.90% +/− 0.27 |
| STK11 | DEL | COSM20871 | P281RfsTer6 | DNA | 0.90% +/− 0.27 |
| KEAP1 | SNP | COSM564847 | R470C | DNA | 0.90% +/− 0.27 |
| KEAP1 | DEL | COSM6916353 | M409* | DNA | 0.90% +/− 0.27 |
| RET | INS | OMINDEL1177 | C634_R635dup | DNA | 0.90% +/− 0.27 |
| RET | DEL | COSM962 | D898_E901del | DNA | 0.90% +/− 0.27 |
| ERBB2 | SNP | COSM48358 | S310F | DNA | 0.80% +/− 0.24 |
| ERBB2 | INS | COSM12558 | Y772_A775dup | DNA | 0.80% +/− 0.24 |
| MET | SNV | COSM6108462 | c.3082 + 1G > T | DNA | 0.80% +/− 0.24 |
| | | | (Splicing − intronic) | | |
| STK11 | SNP | COSM25847 | D194N | DNA | 1.00% +/− 0.30 |
| RET | SNP | COSM133167 | A883S | DNA | 0.75% +/− 0.23 |
| STK11 | INS | OMINDEL1012 | c.598-2_600delAGGCA | DNA | 0.75% +/− 0.23 |
| ERBB2 | CNV | N/A | COPY NUMBER GAIN | DNA | 5.6 +/− 1 copy |
| MET | CNV | N/A | COPY NUMBER GAIN | DNA | 5.6 +/− 1 copy |
| ALK | FUSION | AB374361.1 | EML4- | RNA | 50 +/− 12.5 copies |
| | | | ALK.E6aA20.AB374361.1 | | |
| MET | ALT | N/A | MET-MET.M13M15.1 | RNA | 100 +/− 25 copies |
| | SPLICE | | | | |
| | FORM | | | | |
| NTRK1 | FUSION | COSF1329 | TPM3- | RNA | 50 +/− 12.5 copies |
| | | | NTRK1.T7N10.COSF1329 | | |
| NTRK2 | FUSION | COSF1448 | NACC2- | RNA | 40 +/− 10 copies |
| | | | NTRK2.N4N13.COSF1448 | | |
| NTRK3 | FUSION | N/A | ETV6-NTRK3.E4N14.1 | RNA | 50 +/− 12.5 copies |
| RET | FUSION | COSF1271 | CCDC6- | RNA | 100 +/− 25 copies |
| | | | RET.C1R12.COSF1271 | | |
| ROS1 | FUSION | COSF1197 | SLC34A2- | RNA | 100 +/− 25 copies |
| | | | ROS1.S4R32.COSF1196 | | |

In view of the many possible implementations to which the principles of the disclosure may be applied, it should be recognized that the illustrated examples should not be taken as limiting the scope of the invention. Rather, the scope of the invention is defined by the following claims. We therefore claim as our invention all that comes within the scope and spirit of these claims.

1. A cell-free total nucleic acid composition comprising:
a) genomic DNA comprising fragments of about 75 to about 600 bp in length;
b) one or more DNA molecules comprising one or more variants selected from the group consisting of one or more single nucleotide polymorphisms (SNP), single nucleotide variants, substitution of one or more nucleic acids (e.g., point mutations), insertions (INS), deletions (DEL), premature stop codons, trinucleotide repeats, translocations, inversions, somatic rearrangements, allelomorphs, splice variants, regulatory variants, gene fusions, and copy number variation (CNV), and any combination of two or more thereof; and
c) one or more RNA molecules comprising one or more variants selected from fusions, isoforms, splice variants, and any combination thereof.

2. The cell-free total nucleic acid composition of claim 1, wherein the genomic DNA comprises human genomic DNA.

3. The cell-free total nucleic acid composition of claim 1, wherein the genomic DNA fragments are about 200 bp in length.

4. The cell-free total nucleic acid composition of claim 1, wherein the concentration of the genomic DNA fragments is about 40 to about 60 ng/μL.

5. The cell-free total nucleic acid composition of claim 1, wherein:
at least one of the DNA molecules comprises two or more variants;
at least one of the one or more DNA molecules is present in the composition at a variant allelic frequency of about 0.5% to about 1.5%;
at least one of the one or more DNA molecules is present in the composition at a copy number of about 4 to about 7; and/or
the one or more DNA molecules is selected from the group consisting of plasmid DNA, bacterial artificial chromosome DNA, double-stranded DNA fragments, or any combination of two or more thereof.

6-8. (canceled)

9. The cell-free total nucleic acid composition of claim 5, wherein the double-stranded DNA fragments are gBlock DNA fragments.

10. The cell-free total nucleic acid composition of claim 1, wherein:
at least one of the one or more RNA molecules comprises in vitro transcribed RNA; and/or
at least one of the RNA molecules is present in the composition at a copy number of about 25 to about 125 copies.

11. (canceled)

12. The cell-free total nucleic acid composition of claim 1, wherein at least one of the DNA variants, at least one of the RNA variants, or both comprise a variant associated with a disease or disorder.

13. The cell-free total nucleic acid composition of claim 12, wherein the disease or disorder is cancer.

14. The cell-free total nucleic acid composition of claim 1, further comprising an aqueous solution.

15. The cell-free total nucleic acid composition of claim 14, wherein the aqueous solution is a nucleic acid dilution solution.

16. A kit comprising the cell-free total nucleic acid composition of claim 1.

17. The kit of claim 16, further comprising one or more enzymes, buffers, oligonucleotide primers, oligonucleotide adaptors, oligonucleotide probes, or any combination of two or more thereof.

18. A method of making a cell-free total nucleic acid composition comprising combining:
a) a population of genomic DNA fragments of about 75 to about 600 bp in length;
b) one or more DNA molecules comprising one or more variants selected from the group consisting of one or more single nucleotide polymorphism (SNP), single nucleotide variants, substitution of one or more nucleic acids (e.g., point mutations), insertion (INS), deletion (DEL), premature stop codon, trinucleotide repeat, translocation, inversion, somatic rearrangement, allelomorph, splice variant, regulatory variant, gene fusion, copy number variation (CNV), and any combination of two or more thereof; and
c) one or more RNA molecules comprising one or more variants selected from fusions, isoforms, and any combination thereof.

19. The method of claim 18, wherein the population of genomic DNA fragments is prepared by:
fragmenting a preparation of total genomic DNA; and
purifying fragments of about 75 to about 600 bp in length.

20. The method of claim 18, wherein:
the genomic DNA fragments are about 200 bp in length;
the genomic DNA is human genomic DNA; and/or
the one or more DNA molecules comprise one or more of plasmid DNA, bacterial artificial chromosome (BAC) DNA, double-stranded DNA fragments, or any combination of two or more thereof.

21-22. (canceled)

23. The method of claim 20, further comprising:
fragmenting one or more of the plasmid DNA or BAC DNA; and
purifying fragments of about 100 to about 500 bp in length.

24. The method of claim 18, further comprising:
preparing the one or more RNA molecules comprising the one or more variants by in vitro transcription from one or more DNA molecules encoding the RNA.

25. The method of claim 18, further comprising:
measuring the concentration of the genomic DNA fragments, the one or more DNA molecules, and/or the one or more RNA;
determining the copy number of the genomic DNA fragments, the one or more DNA molecules, and/or the one or more RNA; or
both.

26. The method of claim 18, wherein the combining comprises:
mixing each of the genomic DNA fragments, the one or more DNA molecules, and/or the one or more RNA molecules at a selected concentration or copy number; and/or
combining the genomic DNA fragments, the one or more DNA molecules, and the one or more RNA molecules in an aqueous solution.

27. (canceled)
